(19) United States
(12) Patent Application Publication
(10) Pub. No.: US 2019/0098887 A1
(43) Pub. Date: Apr. 4, 2019

(54) PARASITIC ARTHROPOD MITIGATION DEVICE AND METHOD OF USE THEREOF

(71) Applicant: Jason A. Dean, Kirkville, NY (US)

(72) Inventor: Jason A. Dean, Kirkville, NY (US)

(21) Appl. No.: 16/149,022

(22) Filed: Oct. 1, 2018

Related U.S. Application Data
(60) Provisional application No. 62/606,623, filed on Sep. 30, 2017.
(60) Provisional application No. 62/683,558, filed on Jun. 11, 2018.

(51) Int. Cl. A01M 3/00; A61L 2/00; A61L 2/26

(52) U.S. Cl. CPC A01M 3/005; A01K 67/033; A61L 2/26; A61L 2/0047

(57) ABSTRACT

A method and apparatus for the collection of parasitic arthropods. In one specific example, the parasitic arthropod is a tick, which prefers to cling to the tops of foliage, while waiting for a passing host. The apparatus collects the tick. In one example the apparatus uses a suction force to collect the tick. In other examples, cloth designed to have a tick attach itself thereto is passed over a surface where ticks are believed to be present. In some cases the apparatus further comprises an irradiation apparatus. The irradiation apparatus exposes the ticks to a UVC radiation source, designed to disrupt both the life-cycle of the parasites, as well as to eliminate potential blood-borne pathogens that the ticks may be carrying. In one example, the blood-borne illness is Lyme disease.

### CROSS-REFERENCE TO RELATED APPLICATIONS

This application claims priority to and the benefit of co-pending U.S. provisional patent application Ser. No. 62/606,623, filed Sep. 30, 2017, and priority to and the benefit of co-pending U.S. provisional patent application Ser. No. 62/683,558, filed Jun. 11, 2018, each of which applications is incorporated herein by reference in its entirety.

### FIELD OF THE INVENTION

The invention relates to mitigation of arthropods in general and particularly to systems and methods for mitigating arthropods without application of chemical agents.

### BACKGROUND OF THE INVENTION

The Centers for Disease Control (CDC) has recently reported that illnesses from mosquito, tick, and flea bites have tripled in the U.S., with more than 640,000 cases reported during the 13 years from 2004 through 2016. Nine new germs spread by mosquitoes and ticks were discovered or introduced into the United States during this time. See “Illnesses on the Rise” Vital Signs, CDC News Release dated May 1, 2018, copy appended hereto.

This is CDC's first summary collectively examining data trends for all nationally notifiable diseases caused by the bite of an infected mosquito, tick, or flea. It provides detailed information on the growing burden of mosquito-borne and tickborne illnesses in the U.S.

Zika, West Nile, Lyme, and chikungunya—a growing list of diseases caused by the bite of an infected mosquito, tick, or flea—have confronted the U.S. in recent years, making a lot of people sick.

In the United States, some ticks carry pathogens that can cause human disease, including:

Anaplasmosis is transmitted to humans by tick bites primarily from the blacklegged tick (Ixodes scapularis) in the northeastern and upper midwestern U.S. and the western blacklegged tick (Ixodes pacificus) along the Pacific coast.

Babesiosis is caused by microscopic parasites that infect red blood cells. Most human cases of babesiosis in the U.S. are caused by Babesia microti. Babesia microti is transmitted by the blacklegged tick (Ixodes scapularis) and is found primarily in the northeast and upper midwest.

Borrelia mayonii infection has recently been described as a cause of illness in the upper midwestern United States. It has been found in blacklegged ticks (Ixodes scapularis) in Minnesota and Wisconsin. Borrelia mayonii is a new species and is the only species besides B. burgdorferi known to cause Lyme disease in North America.

Borrelia miyamotoi infection has recently been described as a cause of illness in the U.S. It is transmitted by the blacklegged tick (Ixodes scapularis) and has a range similar to that of Lyme disease.

Bourbon virus infection has been identified in a limited number patients in the Midwest and southern United States. At this time, we do not know if the virus might be found in other areas of the United States.

Colorado tick fever is caused by a virus transmitted by the Rocky Mountain wood tick (Dermacentor andersoni). It occurs in the Rocky Mountain states at elevations of 4,000 to 10,500 feet.

Ehrlichiosis is transmitted to humans by the lone star tick (Ambylomma americanum), found primarily in the southcentral and eastern U.S.

Heartland virus cases have been identified in the Midwestern and southern United States. Studies suggest that Lone Star ticks can transmit the virus. It is unknown if the virus may be found in other areas of the U.S.

Lyme disease is transmitted by the blacklegged tick (Ixodes scapularis) in the northeastern U.S. and upper midwestern U.S. and the western blacklegged tick (Ixodes pacificus) along the Pacific coast.

Powassan disease is transmitted by the blacklegged tick (Ixodes scapularis) and the groundhog tick (Ixodes cookei). Cases have been reported primarily from northeastern states and the Great Lakes region.

Rickettsia parkeri rickettsiosis is transmitted to humans by the Gulf Coast tick (Amblyomma maculatum).

Rocky Mountain spotted fever (RMSF) is transmitted by the American dog tick (Dermacentor variabilis), Rocky Mountain wood tick (Dermacentor andersoni), and the brown dog tick (Rhipicephalus sangunineus) in the U.S. The brown dog tick and other tick species are associated with RMSF in Central and South America.

START (Southern tick-associated rash illness) is transmitted via bites from the lone star tick (Ambylomma americanum), found in the southeastern and eastern U.S.

Tickborne relapsing fever (TBRF) is transmitted to humans through the bite of infected soft ticks. TBRF has been reported in 15 states: Arizona, California, Colorado, Idaho, Kansas, Montana, Nevada, New Mexico, Ohio, Oklahoma, Oregon, Texas, Utah, Washington, and Wyoming and is associated with sleeping in rustic cabins and vacation homes.

Tularemia is transmitted to humans by the dog tick (Dermacentor variabilis), the wood tick (Dermacentor andersoni), and the lone star tick (Amblyomma americanum). Tularemia occurs throughout the U.S.

364D rickettsiosis (Rickettsia phillipi, proposed) is transmitted to humans by the Pacific Coast tick (Dermacentor occidentalis ticks). This is a new disease that has been found in California.

Mosquitoes are also known to carry diseases. Examples are the Zika virus, West Nile virus; dengue fever and chikungunya.

The only flea-borne disease in the CDC report is plague, the bacterium responsible for the medieval Black Death. It remains rare but persistent: Between two and 17 cases were reported from 2004 to 2016, mostly in the Southwest. The infection can be cured with antibiotics.

The Asian long-homed tick, Haemaphysalis longicomis, is spreading rapidly along the Eastern Seaboard. It has been found in seven states and in the heavily populated suburbs of New York City.

In the prior art, it is known to use various chemical agents either to repel such insects, or to attempt to kill or otherwise render insects harmless. However, many such chemicals pose their own risks to health. In some cases, such applications require people and pets to remain indoors while the chemicals are applied, often by spraying from vehicles, helicopters or airplanes. Use of chemicals also adds costs, which may be significant. Examples of such chemicals are N, N-Diethyl-meta-toluamide (known as DEET), Icaridin, also known as picaridin, and/or ethyl butylacetylarninopropionate (also known as IR3535).

There is a need for systems and methods to render such disease carrying arthropods, particularly ticks, mosquitoes and fleas, harmless, or to mitigate them by removal from a given area, without the expense and difficulties associated with chemical methods.

### SUMMARY OF THE INVENTION

According to one aspect, the invention features a device configured to collect and irradiate a harmful parasitic arthropod, comprising: a mechanical suction module configured to collect an arthropod by sucking in the arthropod entrained in a stream of air; at least one collection module configured to collect and localize the arthropod from the stream of air; and an irradiation source configured to irradiate the arthropod when collected and localized within the at least one collection module.

In one embodiment, the irradiation source comprises a UVC or germicidal irradiation source.

In another embodiment, the irradiation source is configured to render the arthropod harmless.

In yet another embodiment, the irradiation source is configured to kill the arthropod.

In still another embodiment, the irradiation source is configured to render an infectious substance carried by the arthropod harmless.

In a further embodiment, the arthropod is one of a tick a mosquito, and a flea.

According to another aspect, the invention relates to a device configured to collect a harmful parasitic arthropod, comprising: at least one of a mechanical roller and a drag mat; the mechanical roller configured to roll over a region, the region believed to be populated by an arthropod; a cover material applied to an external surface of the mechanical roller, the cover material configured to permit an arthropod to attach itself thereto; the drag mat configured to pass over a region, the region believed to be populated by an arthropod, the drag mat comprising a material configured to permit an arthropod to attach itself thereto; and at least one of a handle and a propulsion module; the handle attached to the at least one of the mechanical roller and the drag mat and configured to allow the at least one of the mechanical roller and the drag mat to be propelled by a user over the region so as to collect the arthropod; the propulsion module attached to the at least one of the mechanical roller and the drag mat and configured to allow the at least one of the mechanical roller and the drag mat to be propelled over the region so as to collect the arthropod.

In one embodiment, the arthropod is one of a tick a mosquito, and a flea.

In another embodiment, the device further comprises at least one collection module configured to collect the arthropod from the cover material of the mechanical roller and to localize the arthropod within the at least one collection module.

In yet another embodiment, the device further comprises an irradiation source configured to irradiate the arthropod when collected and localized within the at least one collection module.

In still another embodiment, the irradiation source comprises a UVC or germicidal irradiation source.

In a further embodiment, the irradiation source is configured to render the arthropod harmless.

In yet a further embodiment, the the irradiation source is configured to kill the arthropod.

In an additional embodiment, the irradiation source is configured to render an infectious substance carried by the arthropod harmless.

According to another aspect, the invention relates to a method of mitigating arthropods, comprising the steps of: providing an apparatus, comprising: a device configured to collect an arthropod from a region of interest and to localize the arthropod in a collection module; and an irradiation source configured to irradiate the arthropod when localized within the collection module; collecting the arthropod; and mitigating the arthropod by subjecting it to radiation from radiation source.

In one embodiment, the arthropod is one of a tick a mosquito, and a flea.

In another embodiment, the irradiation source comprises a UVC or germicidal irradiation source.

In yet another embodiment, the irradiation source is configured to render the arthropod harmless.

In still another embodiment, the irradiation source is configured to kill the arthropod.

In a further embodiment, the irradiation source is configured to render an infectious substance carried by the arthropod harmless.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 depicts parasitic arthropods, such as ticks, clinging to the tops of foliage, whereby an air in-take line is introduced at or around the same elevation, whereby the parasites are subjected to a “suction” force, designed to exceed the gripping strength of the parasites, resulting in the removal and collection thereof of said parasites.

FIG. 2 depicts the transport of the parasitic arthropods, from initial collection, to transport to a holding tank, via a mechanically generated suction force. The parasites are extracted from the intake air by means of an aqueous medium, whereby the resulting air is then exhausted through an exhaust port.

FIG. 3 depicts the operation of a UVC diode array, designed to emit a low frequency wavelength from varying trajectories.

FIG. 4 depicts a cross sectional view illustrating both the collection tank, and the external shielding tank which limits exposure to the UVC light source. Furthermore, the collection tank possesses reflective interior walls, whereby UVC wavelengths are reflective in varying trajectories, and designed to provide maximum exposure of UVC light.

FIG. 5 is an image of a second embodiment configured as a mechanical roller.

FIG. 6 is an image of the interior structure of the roller illustrated in FIG. 5.

FIG. 7 is an image of the interior structure of the roller illustrated in FIG. 6 covered with a synthetic fiber cover that is configured to allow ticks to become attached thereto.

FIG. 8 is an image of the surface of the synthetic fiber cover with a tick attached thereto.

FIG. 9 is an image of a lawn roller, configured to receive arthropods by means of synthetic fibers.

FIG. 10 is an image of a portable unit designed to provide mobility for areas consisting of unmanicured foliage, such as can be found along hiking trails and or off-road applications.

FIG. 11 is an image of the internal irradiation chamber and holding tank.

FIG. 12 is an image of the holding tank housed within the portable containment unit.

FIG. 13 is an image of the topside of the portable containment unit.

FIG. 14 is an image of the right side of the portable containment unit.

FIG. 15 is an image of the backside of the portable containment unit.

FIG. 16 is an image of a third embodiment attached to the front of a riding mower.

FIG. 17 is an image of the third embodiment attached to the front of a riding mower in side view.

FIG. 18 is an image of a commercial filtration tank.

FIG. 19 is an image of the commercial filtration tank and central vacuum system components.

FIG. 20 is an image of the commercial containment housing and filtration tank.

FIG. 21 is an image of the containment housings enclosed within their interlocking hatches.

FIG. 22 is an image of interconnecting hoses configured to supply and remove differing air pressures to the filtration tank.

FIG. 23 is an image of a containment system, mounted to the front of a commercial zero turn mower.

### DETAILED DESCRIPTION

In general, the invention involves providing apparatus and methods by which arthropods are collected, and can be removed from a region of interest, such as a lawn, a playing field, a walking path or trail, and then the arthropods can be neutralized, all of which is accomplished without the deliberate application of hazardous chemicals to the region of interest. The method includes passing a mechanism that may comprise a surface that arthropods preferentially attach themselves to, or a mechanism that provides suction, or both, over the region of interest, allowing the arthropods to attach themselves to the surface or be collected by the suction force, removing the arthropods from the region of interest, and subjecting the arthropods to a neutralizing field such as electromagnetic radiation (UV light, for example). The arthropods may be collected for further analysis by suitable laboratories, for example to count how many (or what percentage of) arthropods are infected with, or are carriers of, various diseases. This can assist health monitoring agencies to determine where (geographically) such infected arthropods are present, and how severe the danger from specific types of arthropods (and the diseases that they are carrying) may be at a given time.

In one embodiment, the present invention comprises an electromechanical device, designed to provide intake pressure, or suction, of parasitic arthropods from grasses, and to collect said arthropods in an aqueous medium, whereby said arthropods are subjected to irradiation, such as from a UVC light source. Said collection system is designed to extract parasitic arthropods from grasses, and to neutralize the harmful bacteria in which they may carry, such as Lyme Borreliosis, Rocky Mountain spotted fever, anaplasmosis, ehrlichiosis, Powassan virus, and or babesiosis; all of which are subject to neutralization through the repeated exposure to an irradiation type light source.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In a first embodiment, a parasitic arthropod collection system, apparatuses, and methods for collecting and neutralizing parasitic arthropods that carry harmful blood-borne pathogens are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or descriptions below.

### First Embodiment

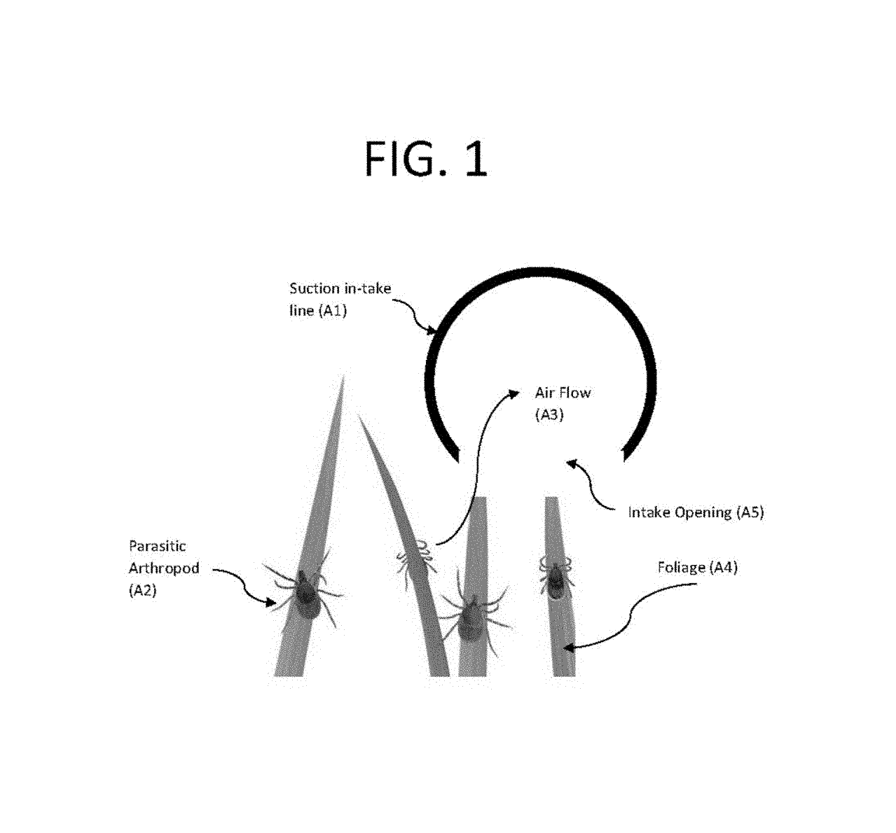

The first embodiment will now be described by referencing the appended figures representing preferred embodiments. FIG. 1 depicts the initial collection of parasitic arthropods. FIG. 1—A1 depicts an intake collection tube, pressurized (or depressurized) so as to generate a suction force (FIG. 1—A3) at its opening (FIG. 1—A5), and designed to remove parasitic arthropods (FIG. 1—A2) from foliage (FIG. 1—A4). The intake is designed to be adjustable in elevation, thereby providing a means to align said intake with the height of the source foliage (FIG. 1—A4).

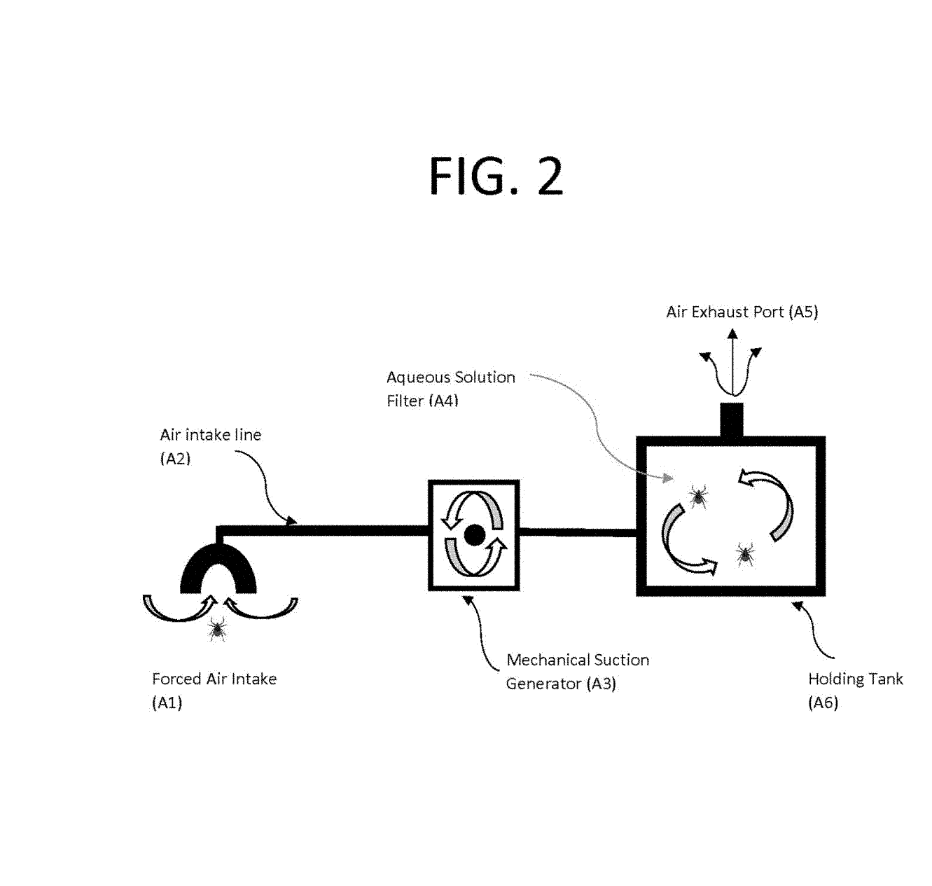

FIG. 2 depicts the processes involved in the collection of parasitic arthropods. The suction force utilized to generate a collective force is generated by mechanical means (FIG. 2—A3), which in turn generates a negative pressure at the intake (FIG. 2—A1). Parasitic arthropods are pulled from the tops of foliage, and are drawn along an intake line (Fig.—A2), and fed to a holding tank (FIG. 2—A6). Pressurized air collected in the holding tank (FIG. 2—A6), is then exhausted through a port (FIG. 2—A5).

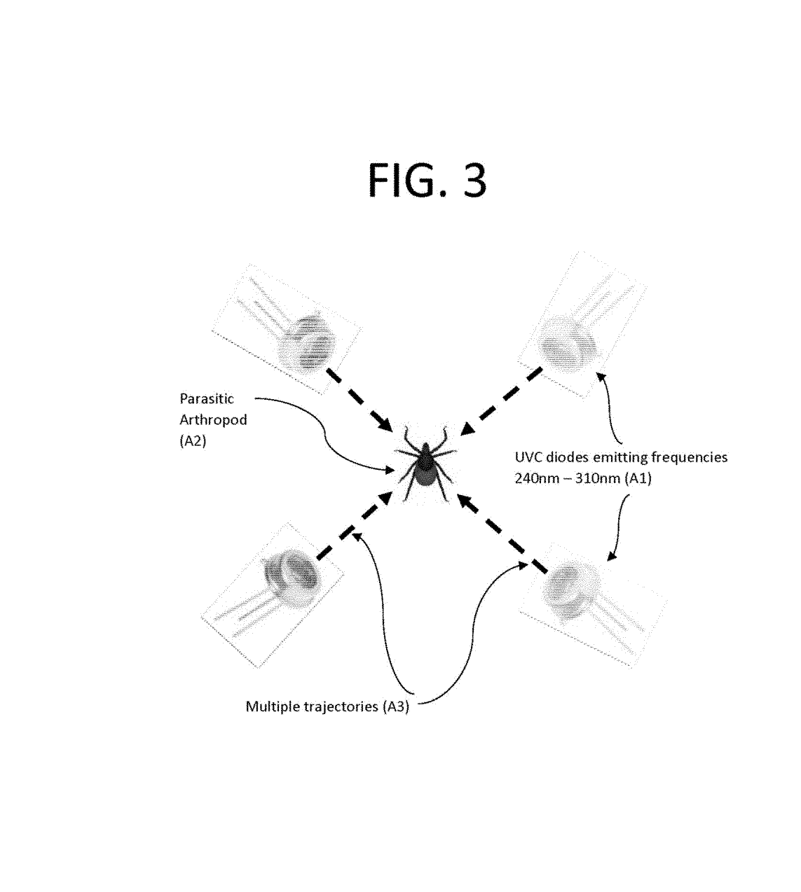

FIG. 3 depicts the primary irradiation source, which involves an array of UVC diodes (FIG. 3—A1), positioned to irradiate parasitic arthropods (FIG. 3—A2) from a multitude of trajectories (FIG. 3—A3). As a result, maximum exposure of UVC radiation (FIG. 3—A1) is achieved.

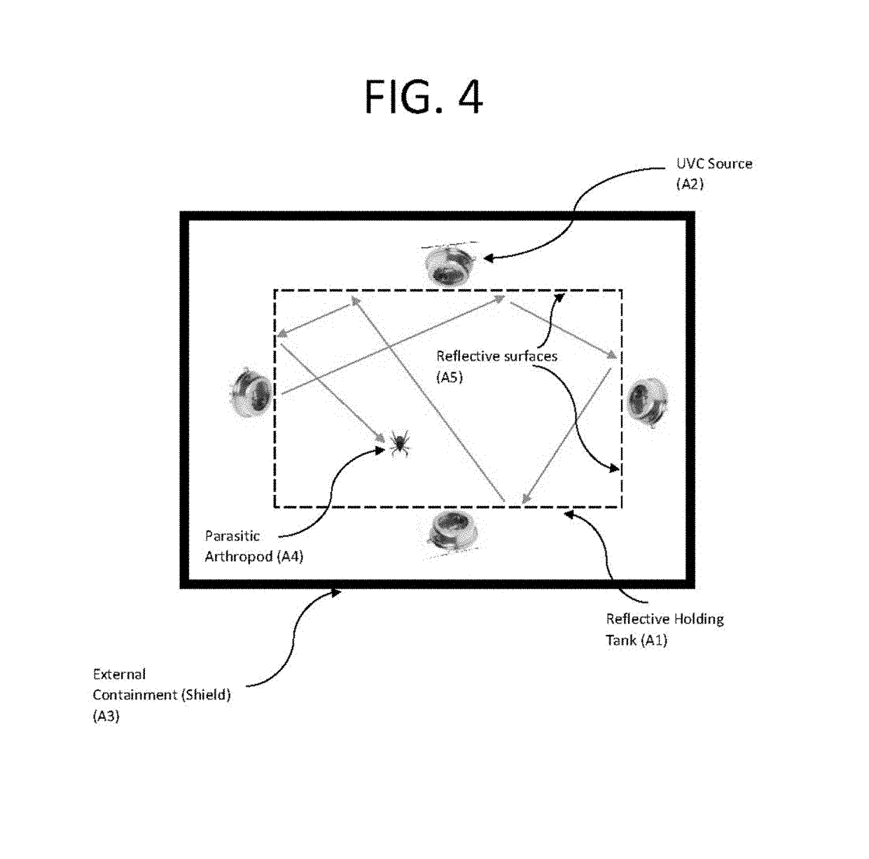

FIG. 4 depicts the composition of the external holding tank (FIG. 4—A3). The external holding tank (FIG. 4—A3) is designed to encapsulate an internal holding tank (FIG. 4—A1), which is designed to hold an aqueous filtering medium, and to provide maximum exposure to UVC radiation (FIG. 4—A2) by means of reflective interior walls (FIG. 4—A5).

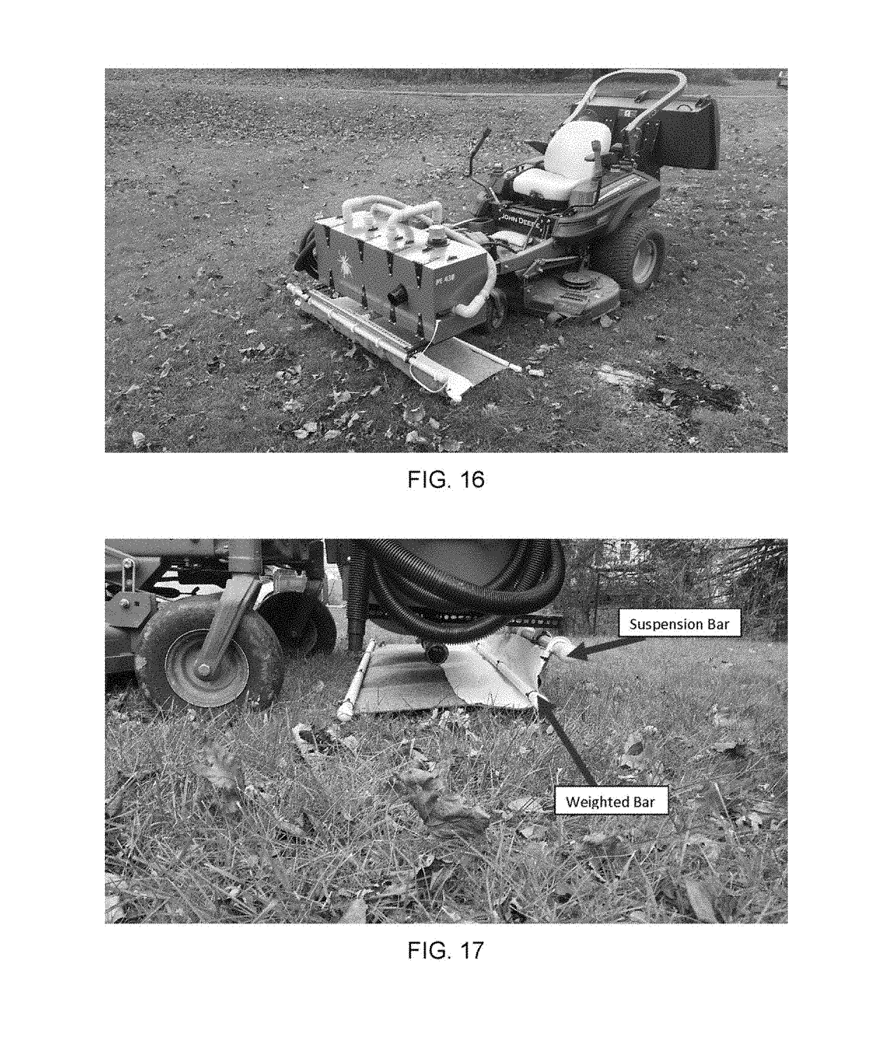

The first embodiment of the invention may be provided as a device that attaches to a mechanical object such as a riding mower, as illustrated in FIG. 16. In the alternative, a smaller model may be provided in the form of a “man pack” that can be worn by a user, with a hose and a vacuum wand as an intake collection tube.

### Second Embodiment

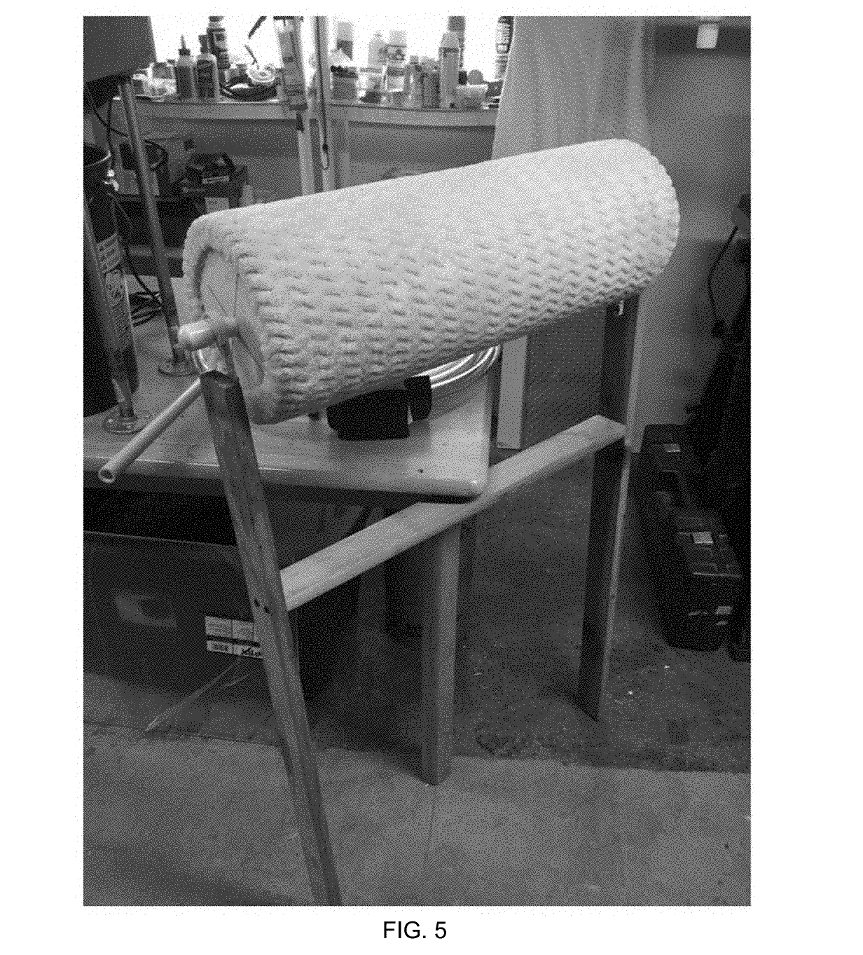

FIG. 5 is an image of a second embodiment configured as a mechanical roller.

The embodiment illustrated in FIG. 5 may be operated by being attached to a mobile device such as a riding lawn mower, or may be operated by being pushed by a human operator, in a manner similar to a push mower.

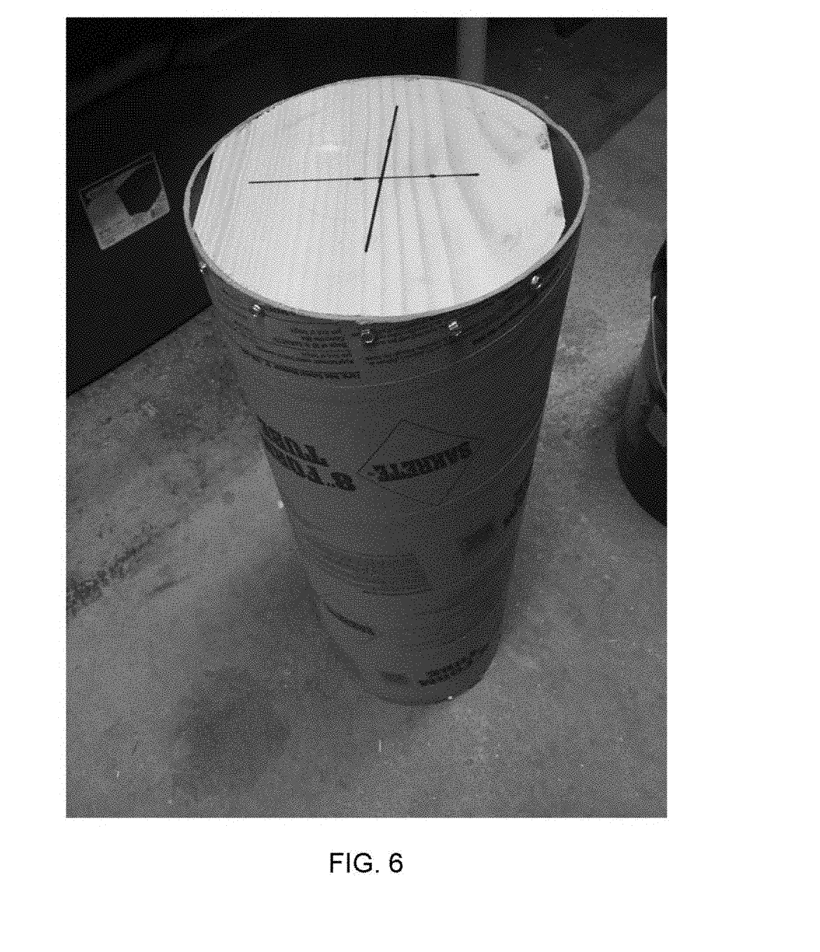

FIG. 6 is an image of the interior structure of the roller illustrated in FIG. 5.

The device of the second embodiment has an interior structure in the form of a cylinder (which may be hollow or may be solid), and having a pair of end plates that can support a rotational structure aligned along the central axis of the cylinder. The rotational structure may be an axle that extends past each end of the cylinder, and may be constructed of a single axle extending from an exterior surface of the first end plate to an exterior surface of the second end plate, or may be two rotational structures, each one attached to a respective exterior surface of each end plate.

The device of the second embodiment has a handle structure that allows each rotational structure, and the attached cylinder, to rotate while the handle structure is used to propel the roller over a surface, such as by way of example, a grass lawn. The roller can be propelled by a human, or by a mechanical device such as a riding mower.

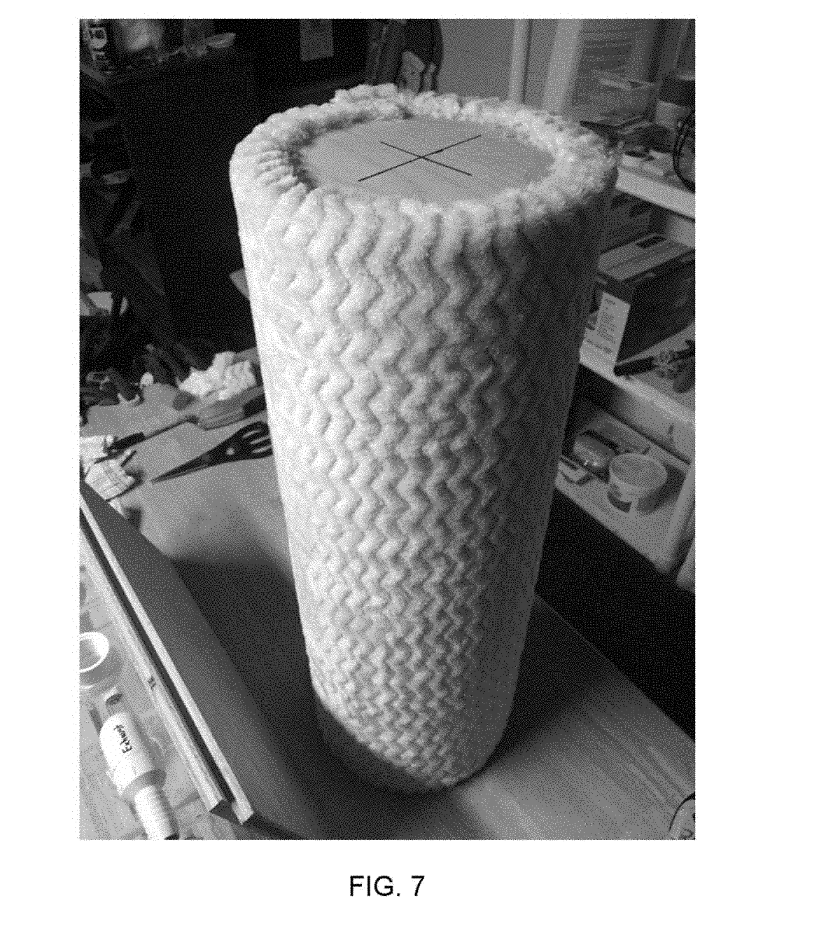

FIG. 7 is an image of the interior structure of the roller illustrated in FIG. 6 covered with a synthetic fiber cover that is configured to allow ticks to become attached thereto.

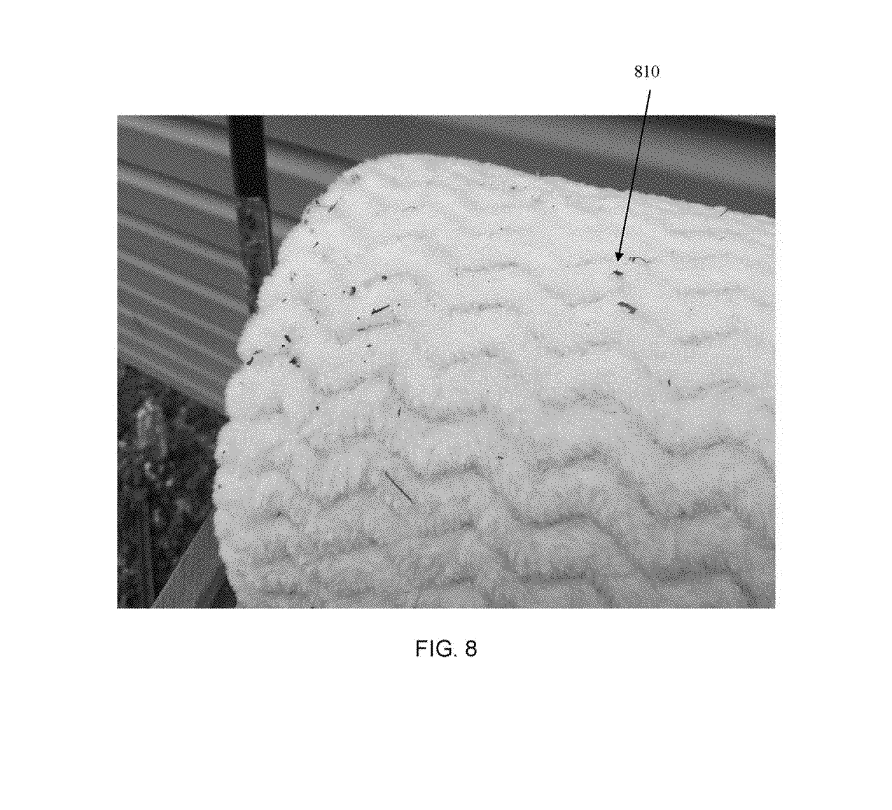

FIG. 8 is an image of the surface of the synthetic fiber cover with a tick 810 attached thereto.

FIG. 9 is an image of a lawn roller, configured to receive arthropods by means of synthetic fibers. The fibers are configured to mimic the characteristics of a host preferred by arthropods. The roller is configured to rotate on top of grass or grasses, so that arthropods will adhere to the synthetic fibers. The rotating action of the roller is designed to prevent the snagging of the synthetic material upon potential debris in the grass.

The roller of FIG. 9 depicts a combination of both roller and drag mat. The combination of both roller and drag mat provide a dual function, so that arthropods that are not successfully snared by the roller are exposed to a secondary entrapment surface. The elevation bars of FIG. 9 provide a way to elevate the mechanism to facilitate the cleaning of both the roller and drag mat. The mechanism is configured to be positioned onto its gripping handles to provide the necessary elevation for cleaning.

Either the roller embodiment such as illustrated in FIG. 5, or the embodiment that incorporates the combination of the roller and the drag mat as illustrated in FIG. 9, collectively described as “the apparatus”, may be propelled by a an animate user (e.g., a person or a work animal) walking behind the apparatus, or by a mechanical device such as a riding mower to which the apparatus is attached.

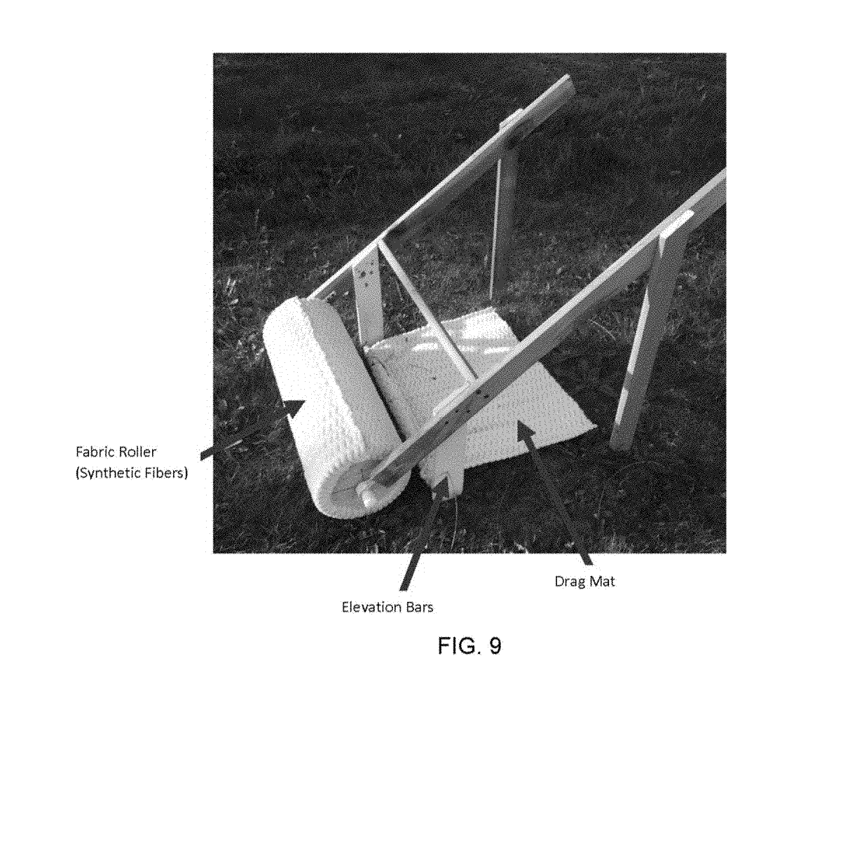

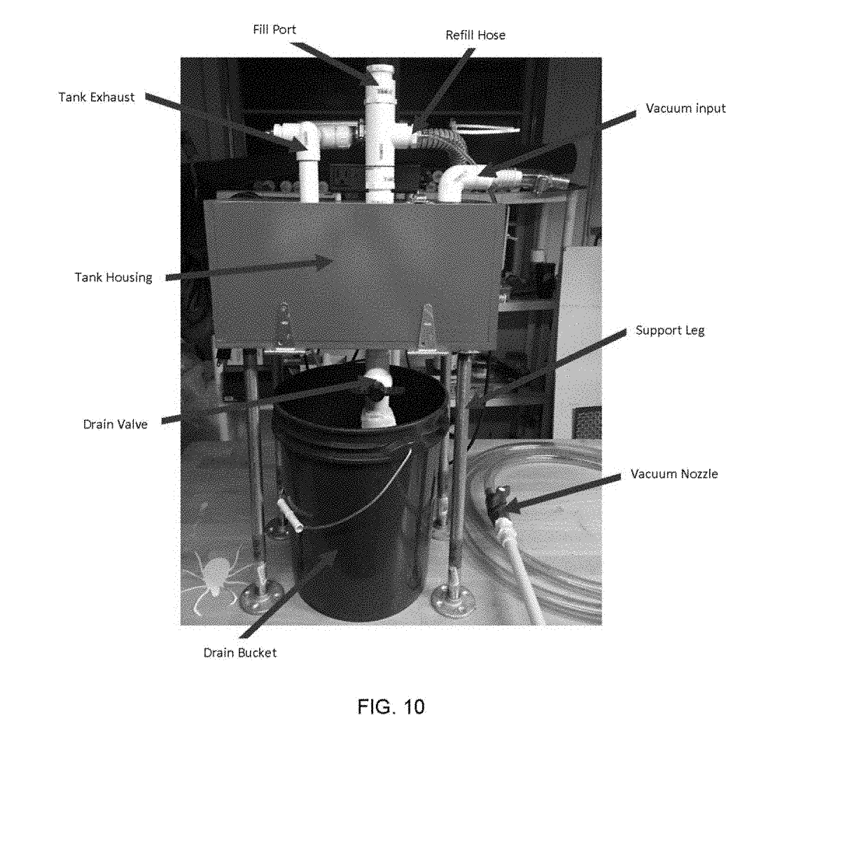

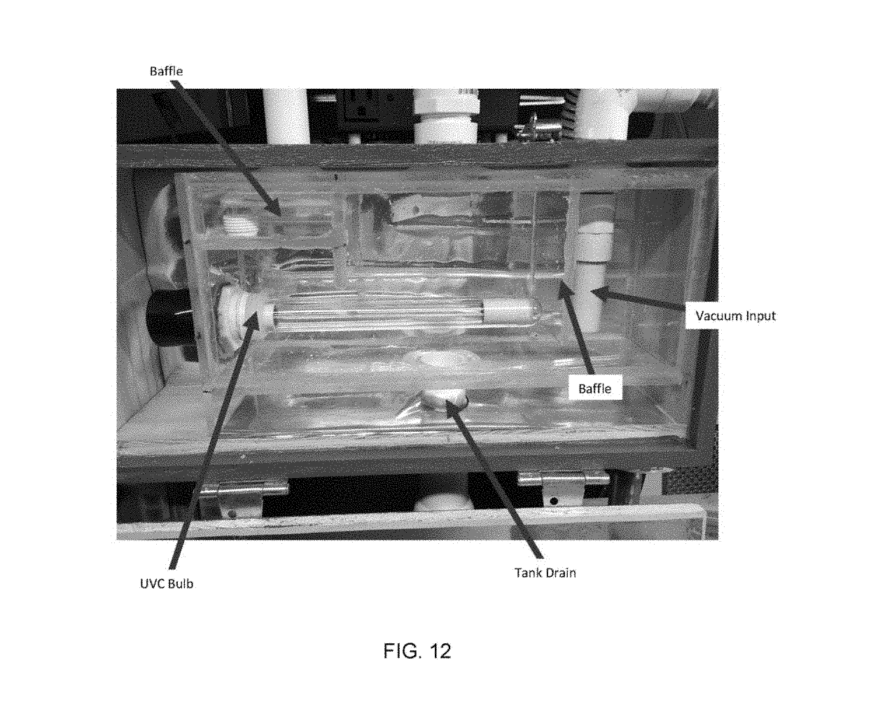

FIG. 10 is an image of a portable unit designed to provide mobility for areas consisting of unmanicured foliage, such as can be found along hiking trails and or off-road applications. The unit of FIG. 10 is configured to be supported by legs to provide elevation requirements to receive a drain bucket. Vacuum pressure is channeled from the tanks exhaust, resulting in a lowered internal pressure in the irradiation tank of FIG. 12, thereby generating a vacuum pressure at the tanks vacuum input of FIG. 10. The resulting vacuum pressure provides the suction force to the vacuum nozzle, channeled through a hose, and which is configured to be received by the vacuum input of FIG. 10.

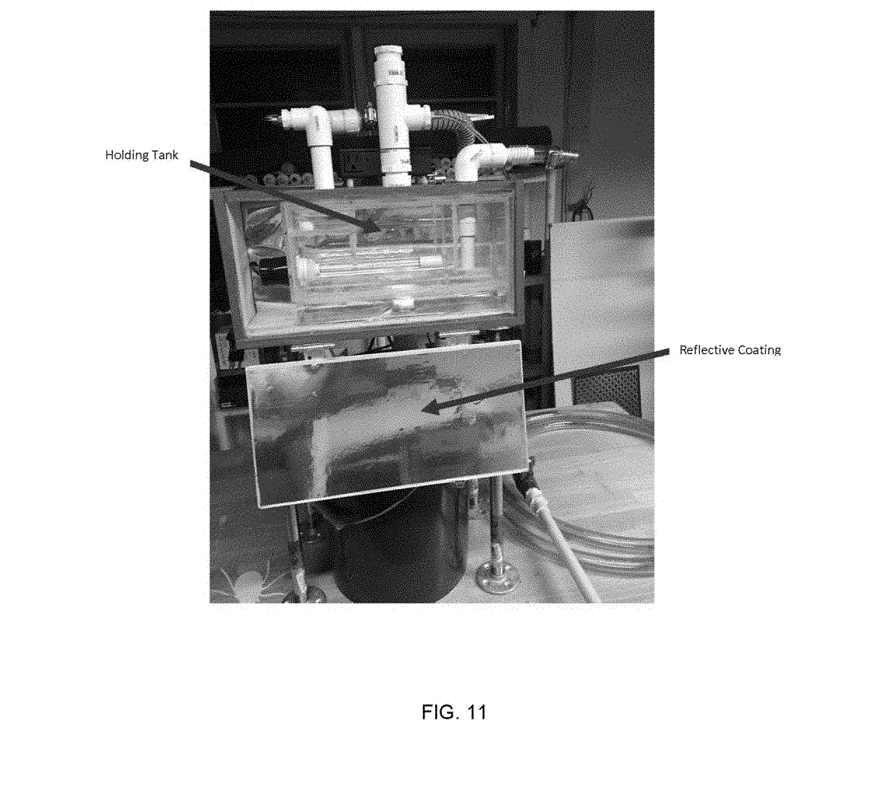

FIG. 11 is an image of the internal irradiation chamber and holding tank. The irradiation source includes a UVC light source configured to irradiate insects held by the filtering medium. Internal lining of the container which houses the tank is configured to reflect irradiation from the UVC light source. The tank includes an acrylic material designed to block 98% of the irradiation source from exiting the holding tank.

FIG. 12 is an image of the holding tank housed within the portable containment unit. The tank is configured to receive a UVC light source internally, designed to provide an irradiation source. The vacuum port of FIG. 12 is configured to be submerged within the filtering medium, and which provides a filtering mechanism by forcing collected air through the liquid medium. The filtering is achieved through surface tension between potential debris and the separating medium. The tank is configured to include baffles designed to manipulate the movement of the separating medium, and the unintentional removal of the medium through vacuum pressure.

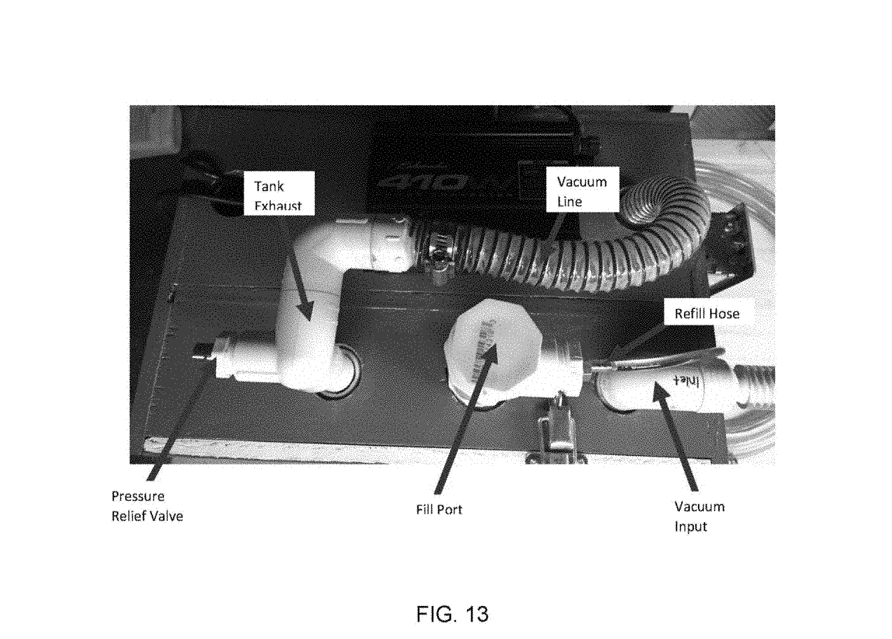

FIG. 13 is an image of the topside of the portable containment unit. Depicted in FIG. 13 is the vacuum input, which provides newly acquired air from a collection hose as a result of vacuum pressure. The vacuum line of FIG. 13 channels the vacuum source from the tank's exhaust after having been passed through the filtering medium. The fill port depicted in FIG. 13 provides a dual function as both the inlet for the liquid medium, as well as a refill inlet designed to introduce additional liquid medium to the tank. The refill hose of FIG. 13 provides the refill source, which is channeled from a separate holding tank. Pressure within the irradiation chamber is configured to be adjusted through a relief valve, designed to provide a variable pressure setting.

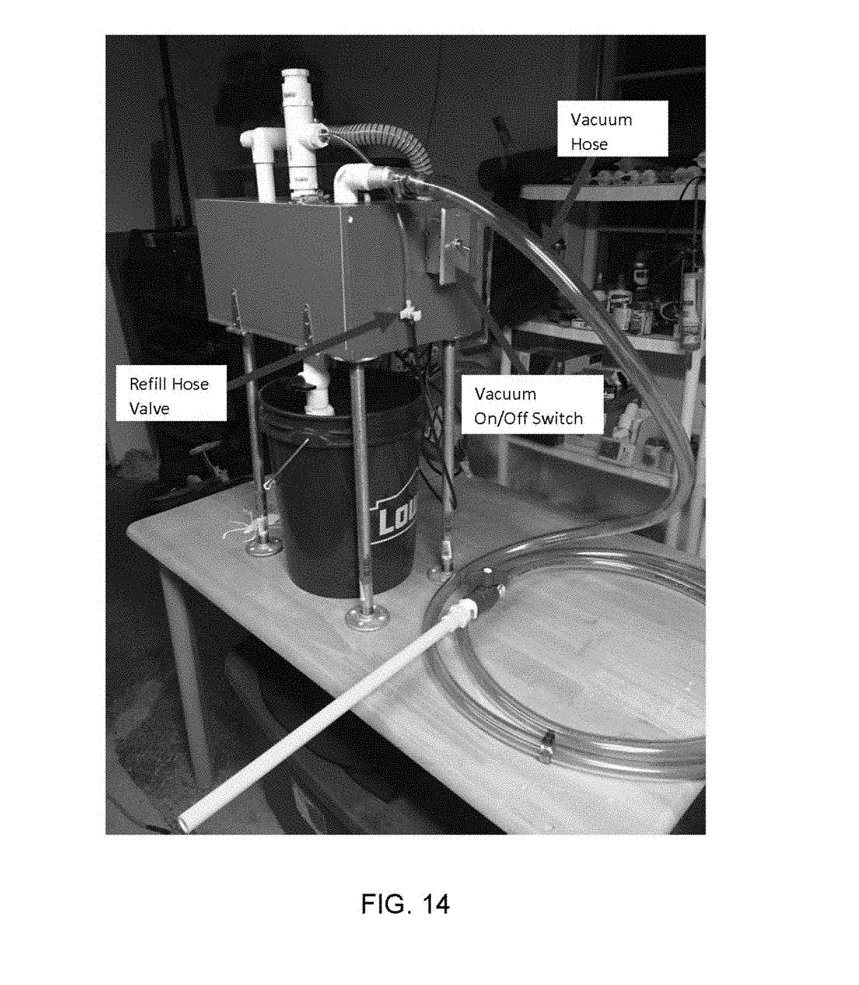

FIG. 14 is an image of the right side of the portable containment unit. The containment unit is configured to receive an additional liquid filtering medium by means of a refill hose, configured to provide flow control by means of a refill hose valve. The vacuum hose of FIG. 14 provides a channel of vacuum pressure to a collection nozzle. The collection nozzle is configured to be received by a hose valve. The hose valve is configured to provide a pressure adjustment at the collection nozzle. Vacuum pressure from the portable containment unit of FIG. 14 is configured to be controlled by an electrical switch.

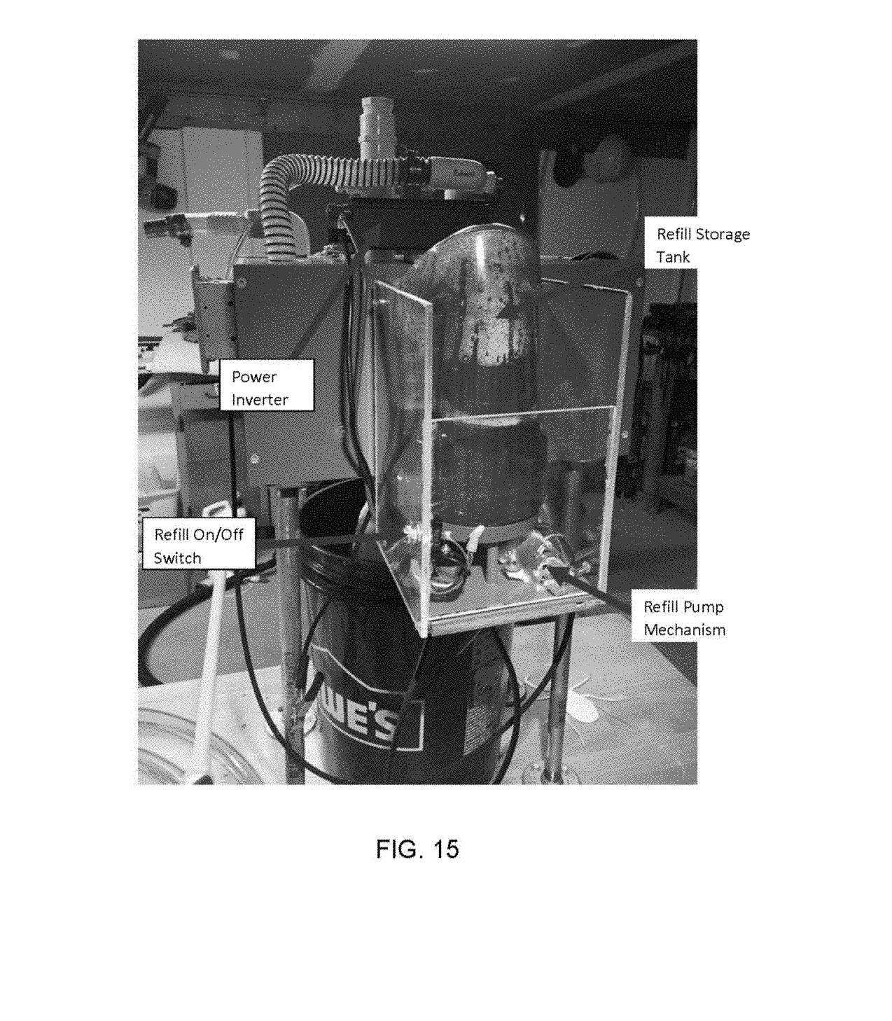

FIG. 15 is an image of the backside of the portable containment unit. The unit is configured to receive additional liquid filtering medium from a refill storage tank. The refill storage tank is configured to be electrically controlled by a power switch. The refill action utilizes a pumping mechanism whereby the liquid medium is channeled along a hose configured to be received by a refill port. In some embodiments, power to the portable containment unit is supplied by a power inverter. The inverter is configured to receive a 12 volt source.

The experimental roller was tested in conjunction with a drag mat. The roller provides a preferred medium for some users, as it allows users to “roll” the grass easily, while avoiding snagging the drag mat on small sticks etc. The observed behavior of the ticks is that they will “hunker down” into the synthetic threads while the roller is moving, and once the roller comes to a stop, they will begin to move about within 2-3 minutes. This movement allows for an easier collection of the ticks, as they are no longer hunkered down in between the threads, but they climb atop of the threads, making them easier to acquire.

The drag mat was attached behind the roller to ensure that ticks were preferentially snared in the roller, and that ticks were not missed. In every use of the roller/mat combination to the present time, 100% of the ticks collected were collected by the roller, and none were found on the drag mat. It is possible that if the number of tick was larger, or that if other arthropods of interest were present, that they might be collected on the drag mat.

### Third Embodiment

FIG. 16 is an image of a third embodiment attached to the front of a riding mower.

FIG. 17 is an image of the third embodiment attached to the front of a riding mower in side view.

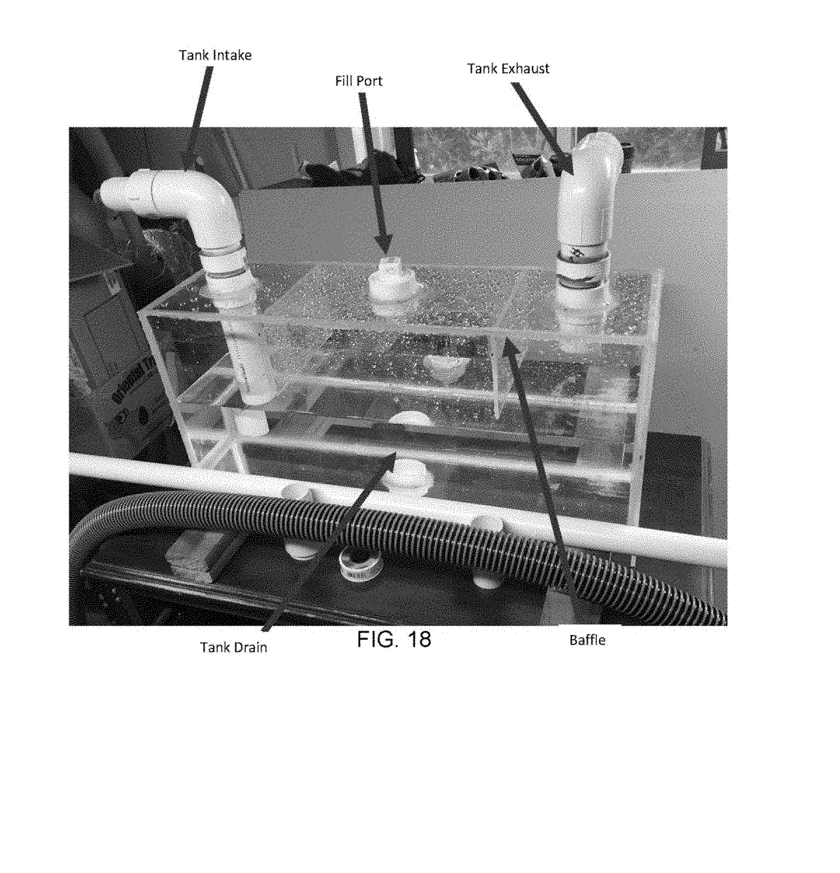

FIG. 18 is an image of a commercial filtration tank. The commercial filtration tank of FIG. 18 includes an air inlet port, fill port, tank drain, and air exhaust port. Vacuum pressure is generated at the exhaust port, thereby reducing the internal pressure of the tank, resulting in air flow at the inlet port as a result of differing pressures. The inlet is configured to receive air from a collection hose, whereby insects will be drawn into the tank as a result of vacuum pressure. The inlet is configured to be below the fill line of the liquid filtering medium, whereby insects are filtered out from the air inlet as a result of surface tension between the insects and the filtering medium. The tank of FIG. 18 is also configured to be emptied of its filtering medium, which exits through a drain plug.

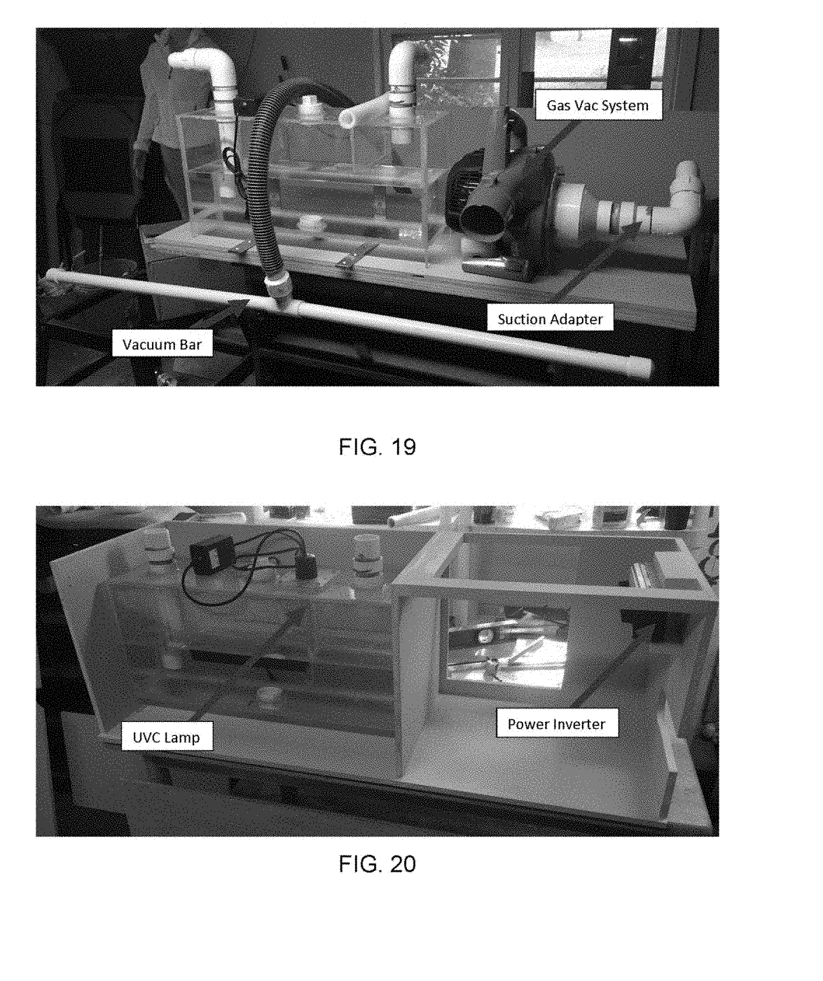

FIG. 19 is an image of the commercial filtration tank and central vacuum system components. In some embodiments, the vacuum pressure required for filtration is generated by a gas powered engine, configured to draw air from the filtration tanks exhaust port. The drawing of air from the exhaust port causes a reduction in air pressure in the tank, resulting in the generation of air flow at the inlet port. The inlet port is configured to receive air from a vacuum bar. The vacuum bar is configured to draw air through a continuous horizontal slit along the bottom of the vacuum bar.

FIG. 20 is an image of the commercial containment housing and filtration tank. The filtration tank is configured to receive multiple UVC light sources. Each of the multiple UVC light sources provides an irradiation source capable of sterilization of both bacteria and viruses. The containment housing of FIG. 20 includes both the containment housing and gasoline powered vacuum housing area. The vacuum housing of FIG. 20 also includes an electrical power inverter, which is configured to receive an electrical supply from an electrical power source, such as a host vehicle.

FIG. 21 is an image of the containment housings enclosed within their interlocking hatches. The air intake of FIG. 21 is configured to generate a low vacuum pressure to the filtration tank, located within the containment housing. The air exhaust of FIG. 21 is configured to exhaust air from the air intake.

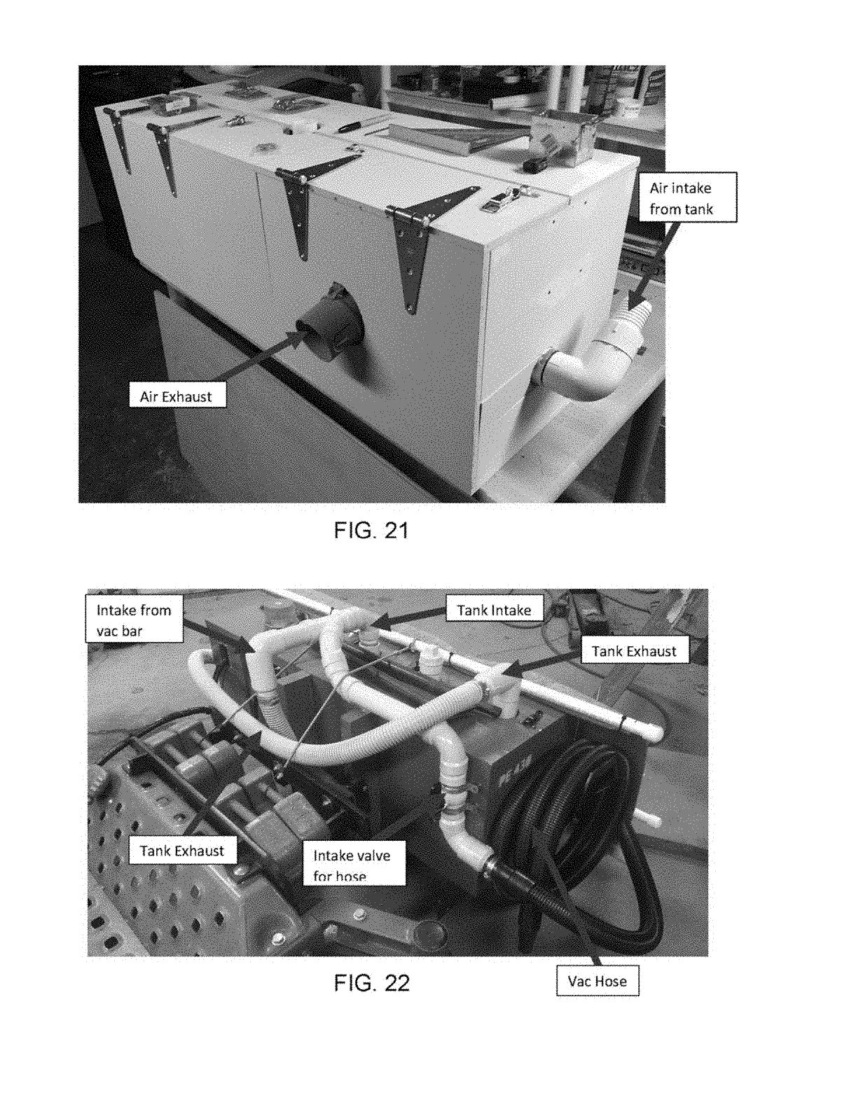

FIG. 22 is an image of interconnecting hoses configured to supply and remove differing air pressures to the filtration tank. The tank exhaust of FIG. 22 is configured to remove air from the filtration tank as a result of vacuum pressure generated from a vacuum source. The air intake of FIG. 22 is configured to supply an air source to the filtration tank. The air source is configured to receive air sources from two separate intakes; (1) a vacuum hose and (2) a vacuum bar. The vacuum bar is configured to be positioned at or around the height of the top of grass or other plant matter.

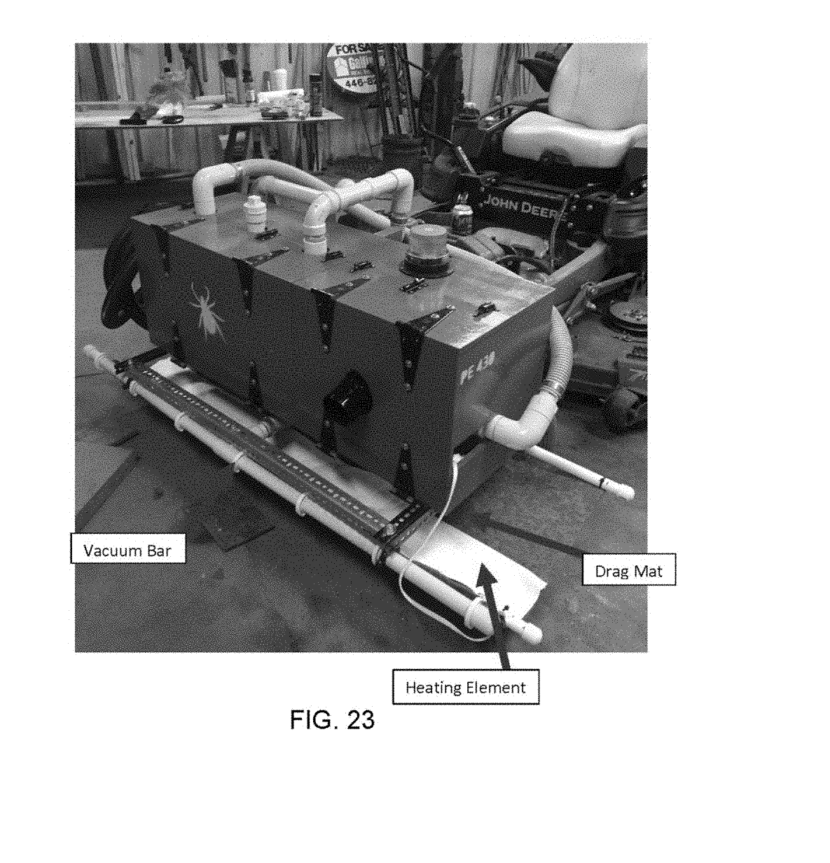

FIG. 23 is an image of a containment system, mounted to the front of a commercial zero turn mower. The vacuum bar of FIG. 23 is configured to be suspended at the height of the top of grass. The vacuum bar is configured to generate a thermal source source (IR), by means of a heating element. The vacuum bar is also configured to provide a suspension source for a drag mat. The drag mat is configured to replicate the characteristics of a preferred host for arthropods.

### Distinction of the Invention Over the Prior Art

Traditional tick collection methods often employ a technique referred to as “tick dragging”, whereby a cloth measuring approximately 1 meter by 1 meter, is used to “drag” along the surface of foliage in an attempt to exploit the arthropods “questing” behavior. The nature of “questing” is to facilitate the adherence to a potential host. However this behavior does not afford one the capacity to differentiate between an actual host, or a synthetic material that has the same physical characteristics of a preferred host. The ticks appendages are configured to establish a “snaring” action via tiny “hook like” tips at the ends of their appendages, which help to facilitate adhesion upon the surface of a host. Although this method of “dragging” has been shown to be an effective means of adhering ticks, its very application places the user in danger of becoming a potential host himself.

In order to drag the cloth, the user either walks directly in front of the cloth, or off to one side. In either configuration, the user potentially places himself directly in the pathway of the ticks.

In the present invention, one can avoid this dilemma by “treating” the foliage (or mitigating the number of arthropods) before the user is required to walk through it. One such device is the “roller”, which is propelled by a user, who applies a force via a handle as the user walks behind the device. The roller's surface is further configured to possess a synthetic covering which mimics the arthropods preferred host. The roller reduces exposure of a user to the likelihood of acquiring an arthropod as a result of causing “adherence” of the tick to the synthetic roller prior to the operator passing through the foliage. In this manner, the foliage or grass is “treated” or “swept” prior to the user walking through the foliage or grass. Arthropods exposed to the synthetic material are “tricked” into believing that it is a suitable host, and as a result, will adhere themselves to the material, thereby reducing potential exposure to an operator.

The “rolling” action of the device further reduces the potential of friction, which the “dragging” method suffers from as it is pulled atop of foliage. By dragging a cloth, it is subject to “snaring” upon debris, such as sticks, for which can reduce its effectiveness. Additionally, the frictional force exhorted upon “clinging” ticks as a result of being dragged across foliage may result in the loss of some of the ticks. By applying a roller, the action reduces the likelihood of “snaring” debris as a result of its spinning action, rather than a frictional drag. The ease by which a user can operate the roller, combined with its reduction in exposure to ticks as a user passes through the foliage, make the roller a preferred collection model.

In each of the embodiments, the invention provides that advantage that the user need not come into contact with a surface that has not been treated to mitigate the number of arthropods.

In some embodiments, such as the first embodiment that uses suction to mitigate the number of arthropods, the intake can be place in a location such that the surface is treated to mitigate the number of arthropods is treated before the user reaches and contacts the treated surface. In some embodiments, such as the second embodiment configured as a mechanical roller, the user can walk behind the roller, so that the surface is treated to mitigate the number of arthropods before the user contacts the treated surface. In some embodiments, such as the third embodiment attached to the front of a riding mower, the user rides on a mechanical device and does not touch the surface that is being treated at all.

### Definitions

Unless otherwise explicitly recited herein, any reference to an electronic signal or an electromagnetic signal (or their equivalents) is to be understood as referring to a non-volatile electronic signal or a non-volatile electromagnetic signal.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A device configured to collect and irradiate a harmful parasitic arthropod, comprising:
a mechanical suction module configured to collect an arthropod by sucking in said arthropod entrained in a stream of air;
at least one collection module configured to collect and localize said arthropod from said stream of air; and
an irradiation source configured to irradiate said arthropod when collected and localized within said at least one collection module.

2. The device of claim 1, wherein the irradiation source comprises a UVC or germicidal irradiation source.

3. The device of claim 1, wherein said irradiation source is configured to render said arthropod harmless.

4. The device of claim 3, wherein said irradiation source is configured to kill said arthropod.

5. The device of claim 3, wherein said irradiation source is configured to render an infectious substance carried by said arthropod harmless.

6. The device of claim 1, wherein said arthropod is one of a tick a mosquito, and a flea.

7. A device configured to collect a harmful parasitic arthropod, comprising:
at least one of a mechanical roller and a drag mat;
said mechanical roller configured to roll over a region, said region believed to be populated by an arthropod;
a cover material applied to an external surface of said mechanical roller, said cover material configured to permit an arthropod to attach itself thereto;
said drag mat configured to pass over a region, said region believed to be populated by an arthropod, said drag mat comprising a material configured to permit an arthropod to attach itself thereto; and
at least one of a handle and a propulsion module;
said handle attached to said at least one of said mechanical roller and said drag mat and configured to allow said at least one of said mechanical roller and said drag mat to be propelled by a user over said region so as to collect said arthropod;
said propulsion module attached to said at least one of said mechanical roller and said drag mat and configured to allow said at least one of said mechanical roller and said drag mat to be propelled over said region so as to collect said arthropod.

8. The device of claim 7, wherein said arthropod is one of a tick a mosquito, and a flea.

9. The device configured to collect a harmful parasitic arthropod of claim 7, further comprising at least one collection module configured to collect said arthropod from said cover material of said mechanical roller or from said drag mat and to localize said arthropod within said at least one collection module.

10. The device configured to collect a harmful parasitic arthropod of claim 9, further comprising an irradiation source configured to irradiate said arthropod when collected and localized within said at least one collection module.

11. The device of claim 10, wherein the irradiation source comprises a UVC or germicidal irradiation source.

12. The device of claim 10, wherein said irradiation source is configured to render said arthropod harmless.

13. The device of claim 10, wherein said irradiation source is configured to kill said arthropod.

14. The device of claim 10, wherein said irradiation source is configured to render an infectious substance carried by said arthropod harmless.

15. A method of mitigating arthropods, comprising the steps of:
providing an apparatus, comprising:
a device configured to collect an arthropod from a region of interest and to localize said arthropod in a collection module; and
an irradiation source configured to irradiate said arthropod when localized within said collection module;
collecting said arthropod; and
mitigating said arthropod by subjecting it to radiation from radiation source.

16. The method of mitigating arthropods of claim 15, wherein said arthropod is one of a tick a mosquito, and a flea.

17. The method of mitigating arthropods of claim 15, wherein the irradiation source comprises a UVC or germicidal irradiation source.

18. The method of mitigating arthropods of claim 15, wherein said irradiation source is configured to render said arthropod harmless.

19. The method of mitigating arthropods of claim 15, wherein said irradiation source is configured to kill said arthropod.

20. The method of mitigating arthropods of claim 15, wherein said irradiation source is configured to render an infectious substance carried by said arthropod harmless.
